(19) United States
(12) Patent Application Publication
(10) Pub. No.: US 2019/0015470 A1
(43) Pub. Date: Jan. 17, 2019

(54) GARLIC COMPOSITIONS

(71) Applicant: INQPHARM GROUP SDN BHD, KUALA LUMPUR (MY)

(72) Inventors: Li Vern PENG, Kuala Lumpur (MY); Patricia DE COSTA, Kuala Lumpur (MY); Zhi Ming BEAH, Kuala Lumpur (MY); Kian Giap LAU, Kuala Lumpur (MY)

(73) Assignee: INQPHARM GROUP SDN BHD, KUALA LUMPUR (MY)

(21) Appl. No.: 16/066,001

(22) PCT Filed: Jan. 6, 2017
(86) PCT No.: PCT/MY2017/000002

§ 371 Date: Jun. 25, 2018

(30) Foreign Application Priority Data
Jan. 8, 2016 (MY) PI 2016700060

(51) Int. Cl. A61K 36/8962; A23L 33/105

(52) U.S. Cl. CPC A61K 36/8962; A61P 19/00; A23L 33/105

(57) ABSTRACT

The present invention relates generally to compositions comprising at least two different garlic extracts, and to the use of said compositions for improving blood flow and/or reducing platelet aggregation and/or maintaining and/or improving the overall health of the circulatory system in a subject. For example, the compositions may also be used to treat or reduce or prevent the onset of one or more of cardiovascular diseases, cerebrovascular or brain diseases, immune diseases, bone, joint and muscle diseases fatigue and cell oxidation. The compositions may also be used to increase energy, improve nutrient delivery and/or metabolic waste removal, as well as to improve cell protection and repairing activities.

### FIELD OF THE INVENTION

The present invention is directed to a composition comprising at least two different garlic extracts, and to the use of said composition for improving (e.g. increasing) blood flow and/or to the use of said composition for reducing platelet aggregation. The composition may be used for maintaining and/or improving the overall health of the circulatory system, for example to treat or reduce or prevent the onset of one or more of circulatory system-associated diseases or disorders and/or to provide beneficial effects to a subject, e.g. the metabolic system of the subject, via the maintenance or improvement of healthy blood flow.

### BACKGROUND OF THE INVENTION

Blood carries all nutrients and oxygen needed by body organs for proper functioning. An adequate amount of blood supply is necessary for one to stay healthy. The blood carries oxygen and nutrients to the target tissues, removes carbon dioxide and metabolic waste products from tissues, and supports the immunological functions, for example by delivering immunogenic cells and proteins to sites of infection. Additionally, blood also helps in regulating body pH and temperature. The concentration of oxygen and other nutrients in cells can be one of the best health indicators and these parameters are closely related to the amount of blood flow (Jayanthy, 2011). Blood flow is thus related to a wide range of health issues, including those associated with the circulatory system.

One factor that may affect blood flow is platelet aggregation. For example, platelet aggregates may adhere to vessel walls, which may cause partial or full blockage of that vessel. This may lead to insufficient blood flow to target organs and thus contribute or result in the development of a particular disease. The level of platelet aggregation may thus be indicative of blood flow.

Alternative and/or improved compositions to maintain or improve (e.g. increase) blood flow and/or to reduce platelet aggregation are therefore desirable.

### SUMMARY OF THE INVENTION

According to a first aspect, there is provided a composition comprising a first garlic extract and a second garlic extract different to the first garlic extract. In certain embodiments, the composition consists essentially of or consists of a first garlic extract and a second garlic extract different to the first garlic extract.

According to a second aspect, there is provided a pharmaceutical composition comprising a composition according to any aspect or embodiment of the invention (e.g. a composition comprising, consisting essentially of or consisting of a first garlic extract and a second garlic extract) and a pharmaceutically acceptable excipient and/or carrier and/or diluent. In certain embodiments, the composition is a nutraceutical composition.

According to a third aspect, there is provided a composition or pharmaceutical composition according to any aspect or embodiment of the invention for use in improving blood flow (e.g. increasing blood flow) in a subject.

According to a fourth aspect, there is provided a composition or pharmaceutical composition according to any aspect or embodiment of the invention for use in reducing platelet aggregation in a subject.

According to a fifth aspect, there is provided a composition or pharmaceutical composition according to any aspect or embodiment of the invention for use in maintaining and/or improving the overall health of the circulatory system in a subject.

According to a sixth aspect, there is provided a use of a composition or pharmaceutical composition according to any aspect or embodiment of the invention in the manufacture of a medicament for improving blood flow (e.g. increasing blood flow) in a subject.

According to a seventh aspect, there is provided a use of a composition or pharmaceutical composition according to any aspect or embodiment of the invention in the manufacture of a medicament for reducing platelet aggregation in a subject.

According to an eighth aspect, there is provided a use of a composition or pharmaceutical composition according to any aspect or embodiment of the invention in the manufacture of a medicament for maintaining and/or improving the overall health of the circulatory system in a subject.

According to a ninth aspect, there is provided a therapeutic method for improving blood flow (e.g. increasing blood flow) in a subject, comprising administering a composition or pharmaceutical composition according to any aspect or embodiment of the invention to the subject.

According to a tenth aspect, there is provided a therapeutic method for reducing platelet aggregation in a subject, comprising administering a composition or pharmaceutical composition according to any aspect or embodiment of the invention to the subject.

According to an eleventh aspect, there is provided a therapeutic method for improving and/or maintaining the overall health of the circulatory system of a subject, comprising administering a composition or pharmaceutical composition according to any aspect or embodiment of the invention to the subject.

According to a twelfth aspect, there is provided a non-therapeutic method for maintaining or improving blood flow (e.g. increasing blood flow) in a subject, comprising administering a composition or pharmaceutical composition according to any aspect or embodiment of the invention to the subject.

According to a thirteenth aspect, there is provided a non-therapeutic method for maintaining or reducing platelet aggregation in a subject, comprising administering a composition or pharmaceutical composition according to any aspect or embodiment of the invention to the subject.

According to a fourteenth aspect, there is provided a non-therapeutic method for maintaining and/or improving the overall health of the circulatory system of a subject, comprising administering a composition or pharmaceutical composition according to any aspect or embodiment of the invention to the subject.

According to an fifteenth aspect, there is provided a method for making a composition or pharmaceutical composition according to any aspect or embodiment of the invention, comprising combining a first garlic extract and a second garlic extract and optionally a pharmaceutically acceptable excipient and/or carrier and/or diluent.

Embodiments of the invention will be further described in the detailed description. Any embodiment described herein or any combination of embodiments described herein is applicable to any one or more aspects of the present invention unless clearly contradicted by context.

### BRIEF DESCRIPTION OF THE DRAWINGS

FIG. 1 is a graph showing the platelet aggregation inhibitory effects by different samples of garlic extracts, including deodourized garlic powder extract (IQP-J), black garlic powder extract (IQP-K) and a garlic composition as described in one of the embodiments of the present invention (IQP-A).

### DETAILED DESCRIPTION OF THE INVENTION

The present invention is based on, at least in part, the surprising finding that a combination of different garlic extracts can improve blood flow in a subject. For example, embodiments of the present invention are based on the surprising finding that different garlic extracts may work synergistically to improve blood flow in a subject. Without wishing to be bound by theory, it is believed that the garlic extracts assist in reducing platelet aggregation, which contributes to the improvement in blood flow.

The composition or pharmaceutical composition comprises a first garlic extract and a second garlic extract different from the first garlic extract. The composition or pharmaceutical composition may also further comprise one or more further garlic extracts, which may be different to the first and second garlic extracts. For example, the composition or pharmaceutical composition may further comprise a third garlic extract or further comprise a third and fourth garlic extract or further comprise a third, fourth and fifth garlic extract or further comprise a third, fourth, fifth and sixth garlic extract. In certain embodiments, the composition consists essentially of or consists of the garlic extracts. For example, the composition may consist essentially of or consist of the first garlic extract and the second garlic extract. For example, the composition may consist essentially of or consist of the first garlic extract, the second garlic extract and the third garlic extract.

The term “garlic extract” encompasses aqueous garlic extract, non-aqueous garlic extract (solvent garlic extract), alcoholic garlic extract, garlic concentrate, garlic oil, garlic maceration, garlic powder, garlic granules and any combination of two or more thereof. Hereinafter, the invention may tend to be described in terms of aqueous garlic extracts. However, the invention should not be construed as being limited to such embodiments. Each garlic extract may, for example, be derived from fresh or non-aged garlic. Alternatively, each garlic extract may, for example, be derived from aged garlic.

Each garlic extract may independently be derived from any of the subspecies and varieties of Allium spp., particularly garlic (Allium sativum) that are currently known or are later discovered. Besides, garlic extracts intended to be used in the present composition can also be obtained from other Allium spp., such as Allium ursinum, Allium fistulosum, and Allium tricoccum. For example, each garlic extract may independently be derived from garlic of the subspecies ophioscorodon (hard neck garlic) and sativum (soft neck garlic). For example, each garlic extract may independently be derived from porcelain garlics, rocambole garlics, purple stripe garlics, marbled purple stripe garlics, glazed purple stripe garlics, artichoke garlics, silverskin garlics, asiatic garlics, turban garlics and creole garlics.

Each garlic extract may independently be derived from any form of garlic. For example, each garlic extract may independently be derived from raw garlic, aqueous garlic extract, non-aqueous garlic extract, alcoholic garlic extract, garlic concentrate, garlic oil, garlic maceration, garlic powder or garlic granules. According to one of the embodiments of the present invention, each garlic extract may independently be derived from a garlic that has been treated or processed before the extract is obtained, i.e. aged garlic; or a garlic that has not been treated or processed before the extract is obtained, i.e. fresh or non-aged garlic.

For example, each garlic extract may independently be derived from “aged garlic” or “black garlic”. In general, aged garlic (including black garlic) can be obtained when the garlic bulbs have been stored in a controlled condition and heated under specific temperature, humidity and solvents, for example over several days or weeks, to cause the cloves to darken in colour after undergoing Millard or browning reaction. For example, the type of garlic extract generally known by the term “aged garlic” is obtained by storing the garlic bulbs with alcohol for a few weeks (e.g. 2 weeks) up to about 2 years (e.g. 20 months). Contrarily, the manufacturing process of black garlic does not involve the alcoholic aging step. Black garlic is obtained by storing the garlic bulbs with water for approximately 1 month under relatively high temperature (e.g. greater than 50° C.).

Fresh or non-aged garlic extract refers to extract derived from garlic bulbs without undergoing special treatment or process intentionally to transform or convert its constituents into different compounds. In certain embodiments, each garlic extract may independently be derived from fresh or non-aged garlic that may have been treated or processed by a method other than that used to make “aged garlic” or “black garlic”. For example, the fresh or non-aged garlic extract can be processed or treated to reduce or remove the garlic odour. Such garlic extract is generally known as deodourized garlic extract. Generally, encapsulation or coating process can be applied to mask or reduce the garlic odour. Alternatively, taste-masking ingredients such as green tea, parsley, basil, spinach etc., can be added to mask or reduce the garlic odour in a composition.

Hereinafter, the invention may tend to be discussed in terms of at least one aged garlic extract and at least one non-aged garlic extract. In certain embodiments, the aged garlic extract can be a black garlic extract, caramelised garlic extract and/or fermented garlic extract, for example black garlic powder extract (BGPE). In certain embodiments, the non-aged garlic extract can be deodourized garlic extract, for example deodourized garlic powder extract (DGPE).

Alternatively or additionally, the invention may tend to be discussed in terms of at least one black garlic extract and at least one deodourized garlic extract. For example, the invention may tend to be discussed in terms of at least one black garlic powder extract (BGPE) and at least one deodourized garlic powder extract (DGPE). However, the invention should not be construed as being limited to such embodiments.

The weight ratio of the first garlic extract to the second garlic extract may range from about 1:99 to about 99:1. For example, the weight ratio of the first garlic extract to the second garlic extract may range from about 1:90 to about 90:1, for example from about 1:80 to about 80:1, for example from about 1:75 to about 75:1, for example from about 1:70 to about 70:1, for example from about 1:60 to about 60:1, for example from about 1:50 to about 50:1, for example from about 1:40 to about 40:1, for example from about 1:30 to about 30:1, for example from about 1:20 to about 20:1, for example from about 1:15 to about 15:1, for example from about 1:10 to about 10:1, for example from about 1:9 to about 9:1, for example from about 1:8 to about 8:1, for example from about 1:7 to about 7:1, for example from about 1:6 to about 6:1, for example from about 1:5 to about 5:1, for example from about 1:4 to about 4:1, for example from about 1:3 to about 3:1, for example from about 1:2 to about 2:1.

For example, the first garlic extract may be an aged garlic extract and the second garlic extract may be a non-aged garlic extract and the weight ratio of the first garlic extract to the second garlic extract may range from about 1:10 to about 10:1, for example from about 1:8 to about 8:1, for example from about 1:5 to about 5:1, for example from about 1:3 to about 3:1, for example from about 1:2 to about 2:1. For example, the first garlic extract may be an aged garlic extract and the second garlic extract may be a black garlic extract and the weight ratio of the first garlic extract to the second garlic extract may range from about 1:10 to about 10:1, for example from about 10:1 to about 1:1, for example from about 8:1 to about 1:1, for example about 5:1 to about 1:1, for example from about 3:1 to about 1:1, for example from about 2:1 to about 1:1, for example about 2:1. For example, the first garlic extract may be a non-aged garlic extract and the second garlic extract may be an aged garlic extract and the weight ratio of the first garlic extract to the second garlic extract may range from about 1:10 to about 10:1, for example from about 10:1 to about 1:1, for example about 8:1 to about 1:1, for example about 5:1 to about 1:1, for example from about 3:1 to about 1:1, for example from about 2:1 to about 1:1, for example about 2:1.

For example, the first garlic extract may be BGPE and the second garlic extract may be DGPE and the weight ratio of the first garlic extract to the second garlic extract may range from about 1:10 to about 10:1, for example from about 1:8 to about 8:1, for example from about 1:5 to about 5:1, for example from about 1:3 to about 3:1, for example from about 1:2 to about 2:1. For example, the first garlic extract may be BGPE and the second garlic extract may be DGPE and the weight ratio of the first garlic extract to the second garlic extract may range from about 1:1 to about 10:1, for example from about 1:1 to about 5:1, for example from about 1:1 to about 3:1, for example from about 1:1 to about 2:1, for example about 2:1 or about 7:3.

For example, the first garlic extract may be an aged garlic extract and the second garlic extract may be a DGPE and the weight ratio of the first garlic extract to the second garlic extract may range from about 1:10 to about 10:1, for example from about 1:8 to about 8:1, for example from about 1:5 to about 5:1, for example from about 1:3 to about 3:1, for example from about 1:2 to about 2:1. For example, the first garlic extract may be aged garlic extract and the second garlic extract may be DGPE and the weight ratio of the first garlic extract to the second garlic extract may range from about 1:1 to about 10:1, for example from about 1:1 to about 5:1, for example from about 1:1 to about 3:1, for example from about 1:1 to about 2:1, for example about 2:1 or about 3:7.

In certain embodiments, the first garlic extract may be BGPE and the second garlic extract may be a DGPE and the weight ratio of the first garlic extract to the second garlic extract is 1:2 or 3:7.

Each garlic extract may comprise one or more of allicin, polyphenol, alliin, γ-glutamylcysteine, S-allyl-L-cysteine, other thiosulfinates, sulfur compounds. For example, each garlic extract may comprise all of allicin, polyphenol, alliin, γ-glutamylcysteine and S-allyl-L-cysteine. For example, each garlic extract may comprise all of allicin, polyphenol, alliin, γ-glutamylcysteine, S-allyl-L-cysteine, other thiosulfinates and sulfur compounds. In certain embodiments, the first garlic extract, for example aged garlic extract such as BGPE, contains mainly polyphenol; whereas the second garlic extract, for example non-aged garlic extract such as DGPE, contains mainly allicin. The amount of allicin is standardised based on allicin yield or allicin potential as it is known in the art that allicin is not present in an intact garlic bulb, garlic powder or aqueous garlic powder. It is only present when fresh garlic is crushed or when the garlic powder extract is dissolved in water.

The composition or pharmaceutical composition may comprise one or more of allicin, polyphenol, alliin, γ-glutamylcysteine, S-allyl-L-cysteine, other thiosulfinates, sulfur compounds. For example, the composition or pharmaceutical composition may comprise all of allicin, polyphenol, alliin, γ-glutamylcysteine and S-allyl-L-cysteine. For example, the composition or pharmaceutical composition may comprise all of allicin, polyphenol, alliin, γ-glutamylcysteine, S-allyl-L-cysteine, other thiosulfinates and sulfur compounds.

The composition (e.g. the combination of the garlic extracts, such as the first and second garlic extracts) may comprise equal to or greater than about 0.5% (w/w) allicin. For example the composition (e.g. the combination of the garlic extracts (e.g. the first and second garlic extract components)) may together comprise equal to or greater than about 1.0% (w/w) allicin. For example, the composition (e.g. the combination of the garlic extracts (e.g. the first and second garlic extracts)) may comprise equal to or greater than about 1.5% (w/w), for example equal to or greater than about 2.0% (w/w), for example equal to or greater than about 2.5% (w/w), for example equal to or greater than about 3.0% (w/w), for example equal to or greater than about 3.5% (w/w), for example equal to or greater than about 3.5% (w/w), for example equal to or greater than about 4.0% (w/w), for example equal to or greater than about 4.5% (w/w), for example equal to or greater than about 5.0% (w/w) allicin. For example, the composition (e.g. the combination of the garlic extracts (e.g. the first and second garlic extracts)) may comprise up to about 10.0% (w/w) allicin, for example up to about 8.0% (w/w) allicin, for example up to about 6.0% (w/w) allicin.

The composition (e.g. the combination of the garlic extracts such as the first and second garlic extracts) may comprise equal to or greater than about 0.5% (w/w) polyphenol. For example, the composition (e.g. the combination of the garlic extracts (e.g. the first and second garlic extract components)) may together comprise equal to or greater than about 1.0% (w/w) polyphenol. For example, the composition (e.g. the combination of the garlic extracts (e.g. the first and second garlic extracts)) may comprise equal to or greater than about 1.5% (w/w), for example equal to or greater than about 2.0% (w/w), for example equal to or greater than about 2.5% (w/w), for example equal to or greater than about 3.0% (w/w), for example equal to or greater than about 3.5% (w/w), for example equal to or greater than about 3.5% (w/w), for example equal to or greater than about 4.0% (w/w), for example equal to or greater than about 4.5% (w/w), for example equal to or greater than about 5.0% (w/w) polyphenol. For example, the composition (e.g. the combination of the garlic extracts (e.g. the first and second garlic extracts)) may comprise up to about 10.0% (w/w) polyphenol, for example up to about 8.0% (w/w) polyphenol, for example up to about 6.0% (w/w) polyphenol.

The composition, (e.g. the combination of the garlic extracts such as the first and second garlic extracts) may comprise equal to or greater than about 0.5% (w/w) total thiosulfinates. For example, the composition (e.g. the combination of the garlic extracts (e.g. the first and second garlic extract components)) may together comprise equal to or greater than about 1.0% (w/w) total thiosulfinates. For example, the composition (e.g. the combination of the garlic extracts (e.g. the first and second garlic extracts)) may comprise equal to or greater than about 1.5% (w/w), for example equal to or greater than about 2.0% (w/w), for example equal to or greater than about 2.5% (w/w), for example equal to or greater than about 3.0% (w/w), for example equal to or greater than about 3.5% (w/w), for example equal to or greater than about 3.5% (w/w), for example equal to or greater than about 4.0% (w/w), for example equal to or greater than about 4.5% (w/w), for example equal to or greater than about 5.0% (w/w) total thiosulfinates. For example, the composition (e.g. the combination of the garlic extracts (e.g. the first and second garlic extracts)) may comprise up to about 10.0% (w/w) total thiosulfinates, for example up to about 8.0% (w/w) total thiosulfinates, for example up to about 6.0% (w/w) total thiosulfinates.

The composition, (e.g. the combination of the garlic extracts such as the first and second garlic extracts) may comprise equal to or greater than about 1.5% (w/w) alliin. For example, the composition (e.g. the combination of garlic extracts (e.g. the first and second garlic extract components)) may together comprise equal to or greater than about 2.0% (w/w) alliin. For example, the composition (e.g. the combination of the garlic extracts (e.g. the first and second garlic extracts) may comprise equal to or greater than about 2.5% (w/w), for example equal to or greater than about 2.7% (w/w), for example equal to or greater than about 3.2% (w/w), for example equal to or greater than about 3.5% (w/w), for example equal to or greater than about 4.0% (w/w), for example equal to or greater than about 4.5% (w/w), for example equal to or greater than about 5.0% (w/w), for example equal to or greater than about 5.5% (w/w), for example equal to or greater than about 6.0% (w/w), for example equal to or greater than about 6.5% (w/w), for example equal to or greater than about 7.0% (w/w) alliin. For example, the composition (e.g. the combination of garlic extracts (e.g. the first and second garlic extracts)) may comprise up to about 12.0% (w/w) alliin, for example up to about 10.0% (w/w) alliin, for example up to about 8.0% (w/w) alliin.

The composition, (e.g. the combination of the garlic extracts such as the first and second garlic extracts) may comprise equal to or greater than about 1.5% (w/w) γ-glutamylcysteine. For example, the composition (e.g. the combination of the garlic extracts (e.g. the first and second garlic extract components)) may together comprise equal to or greater than about 2.0% (w/w) γ-glutamylcysteine. For example, the composition (e.g. the combination of the garlic extracts (e.g. the first and second garlic extracts)) may comprise equal to or greater than about 2.5% (w/w), for example equal to or greater than about 3.0% (w/w), for example equal to or greater than about 4.0% (w/w), for example equal to or greater than about 4.5% (w/w), for example equal to or greater than about 5.0% (w/w), for example equal to or greater than about 5.5% (w/w), for example equal to or greater than about 6.0% (w/w), for example equal to or greater than about 6.5% (w/w), for example equal to or greater than about 7.0% (w/w), for example equal to or greater than about 7.5% (w/w) γ-glutamylcysteine. For example, the composition (e.g. the combination of the garlic extracts (e.g. the first and second garlic extracts)) may comprise up to about 12.0% (w/w) γ-glutamylcysteine, for example up to about 10.0% (w/w) γ-glutamylcysteine, for example up to about 8.0% (w/w) γ-glutamylcysteine.

The composition (e.g. the combination of the garlic extracts such as the first and second garlic extracts) may comprise equal to or greater than about 0.3% (w/w) total sulfur. For example, the composition (e.g. the combination of the garlic extracts (e.g. the first and second garlic extract components)) may together comprise equal to or greater than about 0.6% (w/w) total sulfur. For example, the composition (e.g. the combination of the garlic extracts (e.g. the first and second garlic extracts)) may comprise equal to or greater than about 1.0% (w/w), for example equal to or greater than about 1.5% (w/w), for example equal to or greater than about 2.0% (w/w), for example equal to or greater than about 2.5% (w/w), for example equal to or greater than about 3.0% (w/w), for example equal to or greater than about 3.5% (w/w), for example equal to or greater than about 4.0% (w/w), for example equal to or greater than about 4.5% (w/w) total sulfur. For example, the composition (e.g. the combination of the garlic extracts (e.g. the first and second garlic extracts)) may comprise up to about 10.0% (w/w) total sulfur, for example up to about 8.0% (w/w) total sulfur, for example up to about 6.0% (w/w) total sulfur.

The composition (e.g. the combination of the garlic extracts such as the first and second garlic extracts) may comprise equal to or greater than about 0.05% (w/w) S-allyl-L-cysteine. For example, the composition (e.g. the combination of the garlic extracts (e.g. the first and second garlic extract components)) may together comprise equal to or greater than about 0.1% (w/w) S-allyl-L-cysteine. For example, the composition (e.g. the combination of the garlic extracts (e.g. the first and second garlic extracts)) may comprise equal to or greater than about 0.2% (w/w), for example equal to or greater than about 0.3% (w/w), for example equal to or greater than about 0.5% (w/w), for example equal to or greater than about 0.7% (w/w), for example equal to or greater than about 1.0% (w/w), for example equal to or greater than about 1.2% (w/w), for example equal to or greater than about 1.5% (w/w), for example equal to or greater than about 1.7% (w/w), for example equal to or greater than about 2.0% (w/w) S-allyl-L-cysteine. For example, the composition (e.g. the combination of the garlic extracts (e.g. the first and second garlic extracts)) may comprise up to about 10.0% (w/w) S-allyl-L-cysteine, for example up to about 8.0% (w/w) S-allyl-L-cysteine, for example up to about 6.0% (w/w) S-allyl-L-cysteine, for example up to about 5.0% (w/w) S-allyl-L-cysteine.

The composition (e.g. the combination of the garlic extracts such as the first and second garlic extracts) may comprise equal to or greater than about 0.5% (w/w) allicin and/or equal to or greater than about 0.5% (w/w) polyphenol and/or equal to or greater than about 0.5% (w/w) total thiosulfinates and/or equal to or greater than about 1.5% (w/w) alliin and/or equal to or greater than about 1.5% (w/w) γ-glutamylcysteine and/or equal to or greater than about 0.3% (w/w) total sulfur and/or equal to or greater than about 0.05% (w/w) S-allyl-L-cysteine.

The composition (e.g. the combination of the garlic extracts such as the first and second garlic extracts) may comprise equal to or greater than about 0.7% (w/w) allicin and/or equal to or greater than about 0.7% (w/w) polyphenol and/or equal to or greater than about 0.7% (w/w) total thiosulfinates and/or equal to or greater than about 1.6% (w/w) alliin and/or equal to or greater than about 1.5% (w/w) γ-glutamylcysteine and/or equal to or greater than about 0.3% (w/w) total sulfur and/or equal to or greater than about 0.05% (w/w) S-allyl-L-cysteine.

The composition (e.g. the combination of the garlic extracts such as the first and second garlic extracts) may comprise equal to or greater than about 1.5% (w/w) allicin and/or equal to or greater than about 1.5% (w/w) polyphenol and/or equal to or greater than about 1.5% (w/w) total thiosulfinates and/or equal to or greater than about 3.2% (w/w) alliin and/or equal to or greater than about 3.0% (w/w) γ-glutamylcysteine and/or equal to or greater than about 0.6% (w/w) total sulfur and/or equal to or greater than about 0.1% (w/w) S-allyl-L-cysteine.

The composition or pharmaceutical composition may further comprise dietary fibre of plant and/or non-plant origin. The term “dietary fibre” used herein has its normal meaning for this term. It is generally regarded as the indigestible portion of food derived from plants. Typically, there are two main components of dietary fibre: soluble fibre, which dissolves in water, and insoluble fibre, which does not dissolve in water. Soluble fibres include inulin, chitosan, gum acacia, guar gum, low-methoxy and high-methoxy pectin, oat and/or barley beta glucans, carrageenan, psyllium, cyclodextrin, and derivatives thereof. Insoluble fibres include oat hull fibre, pea hull fibre, soy hull fibre, soy cotyledon fibre, sugar beet fibre, cellulose, corn bran and derivatives thereof. The composition may comprise from about 0.1% to about 90% by weight of dietary fibre, for example, from about 1% to about 80% by weight, or from about 1% to about 70% by weight, or from about 1% to about 60% by weight, or from about 1% to about 50% by weight, or from about 5% to about 50% by weight, or from about 10% to about 50% by weight, or from about 20% to about 50% by weight by weight of dietary fibre, based on the total weight of the composition or pharmaceutical composition.

The composition or pharmaceutical composition may further comprise naturally-derived active ingredients such as plant or fruit extracts, for example leaf extracts (e.g. herbs of Curcuma spp. Andrographis spp, etc.), fruit extracts (e.g. melon extracts, mango extracts, grape extracts, etc.), seed extracts (e.g. grape seed extract, guarana extract, etc). In certain embodiments, the composition or pharmaceutical composition may further comprise flavonoids, bioflavonoids (e.g. quercetin, rutinosides) or phytonutrients. Other active ingredients (which may or may not be derived from plant or fruit extracts), which can also be combined with the composition or pharmaceutical composition of the present invention, include chlorella, collagen spirulina, hyaluronic acid, CoQ-10, plant sterol, beta glucan, red yeast rice, resveratrol, astaxanthin, lutein, glutathione, anthocyanidin, cranberry, bilberry, blueberry, lycopene, flaxseed, fatty acids, lecithin, melatonin, glucosamine, chondroitin, ashwagandha, asparagus extract, saffron extract, tart cherry powder, lemon verbena extract, capsicum spp., ginseng, green tea extract, beetroot, ginger extract, phosphatidylcholine, rosemary extract, schisandra extract, guava leaf extract, bentonite, ginkgo biloba, amino acids, caffeine, olive extract, goji extract, pomegranate, astragalus, reishi mushroom, bacopa, colostrum, GABA and echinaceae. The composition may comprise from about 0.1% to about 90% by weight of such additional active ingredients, for example, from about 1% to about 80% by weight, or from about 1% to about 70% by weight, or from about 1% to about 60% by weight, or from about 1% to about 50% by weight, or from about 5% to about 50% by weight, or from about 10% to about 50% by weight, or from about 20% to about 50% by weight, based on the total weight of the composition or pharmaceutical composition.

The composition may further comprise other biologically active agents, for example, biologically active agents suitable improving blood flow and/or for reducing platelet aggregation and/or for treating or preventing any disease associated with blood flow. For example, the biologically active agent may be selected from the group consisting of anticoagulants (e.g. heparin or warfarin), antiplatelet drugs (e.g. aspirin), cholesterol-lowering medications, high blood pressure medications, medications to prevent blood clots and medications to control blood sugar. In certain embodiments, the biologically active agent or agents are present in the composition or pharmaceutical composition in an amount ranging from about 0.001 wt. % to about 50 wt. %, based on the total weight of the composition, for example, about 0.1 wt. % to about 15 wt. %, or from about 0.5 wt. % to about 10 wt. %, or from about 0.5 wt. % to about 5 wt. %, or from about 0.1 wt. % to about 3 wt. %, or from about 0.1 wt. % to about 2 wt. %, or from about 0.1 wt. % to about 1 wt. %, or from about 0.001 wt. % to about 5 wt. %, or from about 0.001 wt. % to about 2 wt. %, or from about 0.001 wt. % to about 1 wt. %, or from about 0.001 wt. % to 20 about 0.5 wt. %, or from about 0.001 wt. % to about 0.1 wt. %, or from about 0.001 wt. % to about 0.01 wt. %.

The composition or pharmaceutical composition may further comprise a nutrient ingredient selected from the group consisting of vitamins and minerals, and combinations thereof. The vitamin may be any one or more of vitamin A, vitamin D, vitamin E, vitamin K, thiamine, riboflavin, pyridoxine, cyanocobalamin, carotenoids (including beta-carotene, zeaxanthin, lutein and lycopene), niacin, folic acid, pantothenic acid, biotin, vitamin C, choline, inositol, and salts and derivatives thereof. The mineral may be any one or more of calcium, phosphorous, magnesium, iron, zinc, manganese, copper, cobalt, boron, iodine, sodium, potassium, molybdenum, selenium, chromium, fluorine and chloride. If present, the composition may comprise from about 0.0001% to about 50% by weight of vitamin(s) and/or mineral(s), based on the total weight of the composition, for example, from about 0.01% to about 45% by weight, from about 0.1% to about 40% by weight, or from about 0.5% to about 30% by weight, or from about 0.5% to about 20% by weight, or from about 0.5% to about 10% by weight, or from about 0.5% to about 5%, or 20 from about 0.5% to about 3%, or from about 0.1% to about 2%, or from about 0.1 to about 1% of vitamin(s) and/or mineral(s), based on the total weight of the composition or pharmaceutical composition. The composition may comprise from about 0.0001% to about 5 wt. %, for example, from about 0.0001% to about 2 wt. %, or from about 0.0001% to about 1 wt. %, or from about 0.0001% to about 0.5 wt. %, or from about 0.0001% to about 0.1 wt. %, or from about 0.0001% to about 0.01 wt. % by weight of vitamin(s) and/or mineral(s), based on the total weight of the composition or pharmaceutical composition.

The composition of the present invention may be administered in the form of a composition comprising any suitable additional component. The composition may, for example, be a pharmaceutical composition (medicament), suitable for oral, nasal, topical, suppository, intravenous or intradermal administration. The composition may alternatively be a nutraceutical composition, for example, a foodstuff, food supplement, dietary supplement, health supplement, meal replacement product, beverage, beverage supplement, food additive, animal feed or feed additive.

The term “pharmaceutical composition” or “medicament” in the context of this invention means a composition comprising (a pharmaceutically effective amount of) garlic extracts and additionally one or more pharmaceutically acceptable carriers and/or excipients. The pharmaceutical composition may further contain ingredients selected from, for example, diluents, adjuvants, excipients, vehicles, preserving agents, fillers, binders, disintegrating agents, wetting agents, emulsifying agents, suspending agents, sweetening agents, flavouring agents, perfuming agents, antibacterial agents, antifungal agents, lubricating agents, coating agents, encapsulating agents and dispersing agents, depending on the nature of the mode of administration and dosage forms. The pharmaceutical compositions may take the form, for example, of solid preparations including tablets, capsules, caplets, drageés, lozenges, granules, powders, pellets, beads and cachets; and liquid preparations including elixirs, syrups, suspensions, sprays, emulsions, lotions, creams and solutions. Techniques and formulations generally may be found in Remington, The Science and Practice of Pharmacy, Mack Publishing Co., Easton, Pa., latest edition.

In solid dosage forms of the invention for oral administration, the active ingredient(s) may be mixed with one or more pharmaceutically acceptable carriers, such as dicalcium phosphate, and/or any of the following: diluents, fillers or extenders, such as starches, lactose, sucrose, glucose, mannitol, microcrystalline cellulose and/or silicic acid; binders, such as, for example, hydroxypropylcellulose, hypromellose, hydroxypropyl methylcellulose, carboxymethylcellulose, gelatine, polyvinyl pyrrolidones, polyvinyl acetate, sucrose and/or acacia; disintegrating agents, such as starch, for example, potato or tapioca starch, starch derivatives such as sodium starch glycolate, crospolyvinylpyrollidone, calcium carbonate, croscarmellose sodium, alginic acid, and certain silicates; lubricants, such as talc, calcium stearate, magnesium stearate, stearic acid, sodium sulfate stearyl fumarate, solid polyethylene glycols, solubiliser such as sodium lauryl sulfate, flavouring and colouring agents and mixtures thereof.

Tablets, and other solid dosage forms of the pharmaceutical compositions of the invention, may optionally be prepared with coatings and shells, such as enteric coatings and other coatings well known in the pharmaceutical-formulation art. They may also be formulated so as to provide slow or controlled release of the active ingredient(s) therein using, for example, natural and synthetic polymers such as hydroxypropylmethyl cellulose methacrylates, methacrylic acid copolymers (e.g. methyl acrylate-methacrylic acid copolymers and methyl methacrylate-methacrylic acid copolymers), shellac, ethylcellulose, cellulose acetate phthalate, cellulose acetate trimellitate, polyvinyl acetate phthalate, cellulose acetate succinate, hydroxyl propyl methyl cellulose acetate succinate, sodium alginate, waxes, fatty acids, zein, respectively, in varying proportions to provide the desired release profile, other polymer matrices, liposomes and/or microspheres may also be used. These compositions may also optionally contain colourants and/or opacifying agents and may be of a composition such that they release the active ingredient(s) only, or preferentially, in a certain portion of the gastrointestinal tract, optionally, in a delayed manner.

The pharmaceutical compositions may comprise no more than about 50% w/w of pharmaceutically acceptable carrier and/or excipient, for example, no more than about 45% w/w of pharmaceutically acceptable carrier and/or excipients, or no more than about 40% of w/w pharmaceutically acceptable carrier and/or excipients, or no more than about 35% w/w of pharmaceutically acceptable carrier and/or excipients. For example, the pharmaceutical composition may comprise at least about 1% w/w, or at least about 10% w/w, or at least about 15% w/w, or at least about 20% w/w, or at least about 25% w/w, or at least about 30% w/w of pharmaceutically acceptable carrier and/or excipients.

Liquid form preparations include solutions, suspensions, and emulsions, for example, water or water-propylene glycol solutions for oral administration. Liquid preparations can also be formulated in solution in aqueous polyethylene glycol solution. In certain embodiments, the active ingredient(s), i.e., garlic extracts, may be mixed with one or more pharmaceutically acceptable carriers, such as water and/or any of the following: solvent such as propylene glycol, alcohol; humectant such as glycerol; sweeteners such as liquid glucose, corn syrup and sucrose; artificial sweeteners such as aspartame, stevia and sucralose; preservatives such as benzoates and parabens; viscosity modifiers/thickeners such as gums and alginates; buffering agents; flavouring agents and colouring agents.

Also included are solid form preparations, for example, tablets, capsules, granules and powder, which are intended to be converted, shortly before use, to liquid form preparations for oral administration. Such liquid forms include solutions, suspensions, and emulsions. These particular solid form preparations are most conveniently provided in unit dose form and as such are used to provide a single liquid dosage unit. Alternatively, sufficient solid may be provided so that multiple individual liquid doses may be reconstituted when required, by measuring predetermined volumes of the solid form preparation as with a spoon, or other measuring device. The solid form preparations intended to be converted to liquid form may contain, in addition to the active material, flavourings, colourants, stabilizers, buffers, artificial and natural sweeteners, dispersants, thickeners, solubilizing agents, and the like. The liquid utilized for preparing the liquid form preparation may be water, isotonic water, juices, milk, ethanol, and the like as well as mixtures thereof.

The terms “food”, “foodstuff”, “food supplement”, “dietary supplement”, “health supplement”, “meal replacement product”, “beverage” and “beverage supplement” used herein have the normal meanings for those terms, and are not restricted to pharmaceutical preparations. Other composition forms are also included within the present invention. These may, for example, include, a foodstuff precursor such as a rehydratable powder or a beverage precursor such as a powder dispersible in water, milk or other liquid.

Also included are solid form preparations which are intended to be combined with a food or foodstuff before oral consumption. The solid form preparations may be mixed into the food or foodstuff or applied to the food or foodstuff, e.g., by sprinkling onto the food or foodstuff. Such solid forms include powders, granules, pellets and the like. Such food of foodstuffs include, without limitation, prepared meals (cooked or fresh), soup, dairy based products (e.g., yoghurt, cream, crème-fraîche), flour based products such as bread and pasta, snack or convenience items such as snack bars (e.g., chocolate bars), confectionary products, and the like.

In certain embodiments, the food or foodstuff, and the like, comprises from about 0.1 wt. % to about 50 wt. % of the composition of the invention described herein, based on the total weight of the food or foodstuff, for example, from about 0.1 wt. % to about 40 wt. %, or from about 0.1 wt. % to about 30 wt. %, or from about 0.1 wt. % to about 20 wt. %, or from about 0.1 wt. % to about 15 wt. %, or from about 0.1 wt. % to about 10 wt. %, or from about 0.1 wt. % to about 8 wt. %, or from about 0.1 wt. % to about 6 wt. %, or from about 0.1 wt. % to about 4 wt. %, or from about 0.1 wt. % to about 2 wt. % of the composition of the invention described herein. In certain embodiments, the food or foodstuff, and the like, comprise at least about 0.2 wt. % of the compositions of the invention described herein, based on the total weight of the food or foodstuff, for example, at least about 0.5 wt. %, or at least about 1 wt. %, or at least about 5 wt. % of the composition of the invention described herein.

In certain embodiments, the composition is orally administered daily to the subject. Without wishing to be bound by theory, it is believed that the composition improves blood flow, for example by reducing platelet aggregation.

The amount of composition administered may be varied depending upon the requirements of the subject. For both therapeutic and non-therapeutic applications, the amount of composition administered may be varied depending upon the desired results, the requirements of the subject and the severity of the condition being treated. Determination of the proper amount/dosage for a particular situation is within the skill of the art. For example, for therapeutic applications a physician or veterinarian having ordinary skill in the art can readily determine and prescribe the effective amount of the pharmaceutical composition required. The total daily amount/dosage may be divided and administered in portions during the day if desired.

In general, a suitable daily dose of active agents in the composition according to the invention will be that amount which is the lowest dose effective to produce the desired effect, for example, a therapeutic effect, and/or to improve (e.g. increase) blood flow and/or to reduce platelet aggregation. It is contemplated that a wide range of doses may be used, due to the non-toxic nature of the composition. A person of ordinary skill in the art will understand that a suitable dose or dosage will typically vary from subject to subject, and will dependent on factors such as the blood flow and/or the severity of health conditions of the subject at the outset of administration of the composition. For example, a subject seeking to maintain a healthy blood flow and/or platelet aggregation level may need to consume a lesser amount of the composition than subject seeking to improve their blood flow and/or platelet aggregation level. For example, the dose of active agents (i.e. garlic extracts) in the composition may be up to 15 g per day, for example, up to about 10 g per day, or up to about 5 g per day. In certain embodiments, the doses of active agents in the composition is in the range of 100 mg to about 3 g per day, which may be administered as two or three or more sub-doses administered separately at appropriate intervals throughout the day, optionally in unit dosage forms. In certain embodiments, the dose of active agents in the composition may be from about 200 mg to about 3 g of each garlic extract component per day, for example, from about 500 mg to about 3 g of each component per day, or from about 750 mg to about 2.5 g of each component per day, or from about 1000 mg to about 2000 mg of each component per day. In certain embodiments, the composition may be administered two or three times a day, optionally before, with, or after a meal. In certain embodiments, each dose of active agents is no more than about 5 g, for example, no more than about 3 g, for example, no more than about 2.5 g. Each dose of the garlic extracts in the composition may be combined with other conventional agents for maintaining and/or improving blood flow and/or platelet aggregation levels.

The compositions and pharmaceutical compositions described herein may be used in various therapeutic and non-therapeutic applications. For example, the compositions and pharmaceutical compositions described herein may be used to provide one or more beneficial effects to a patient. For example, the compositions and pharmaceutical compositions described herein may be used to improve blood flow and/or to reduce platelet aggregation. For example, the compositions and pharmaceutical compositions described herein may be used to increase blood flow. For example, the compositions and pharmaceutical compositions described herein may be used in an in vitro method or in an in vivo method. The methods may comprise administering the composition or pharmaceutical composition described herein to a subject.

In certain embodiments, the composition may be used for maintaining and/or improving the overall health of the circulatory system, for example to treat or reduce or prevent the onset of one or more circulatory system-associated diseases or disorders, and/or to provide beneficial effects to the metabolic system via the maintenance or improvement of healthy blood flow. This may, for example, involve providing more than one of the beneficial effects described herein.

An adequate amount of blood supply to a physiological system is necessary for one to stay healthy. The blood carries oxygen and nutrients to the target tissues, removes carbon dioxide and metabolic waste products from tissues, and supports the immunological functions of the system. Additionally, blood also helps in regulating body pH and temperature. Concentration of oxygen and other nutrients in cells can be one of the best health indicators and these parameters are closely related to the amount of blood flow.

By having an improved blood flow in the physiological system, in which nutrient delivery to cells or tissues is improved, the compositions described herein may also be used to improve or promote cardiovascular or heart health, including cholesterol control, and blood pressure control.

The compositions described herein may also be used for improving cerebrovascular or brain health, such as to improve focus, concentration and memory support, via the maintenance of healthy blood flow.

In certain embodiments, the compositions described herein may also be used as an immune booster. For example, the compositions can be used as an anti-infection, anti-inflammation and antioxidant (which provides cell protection).

In certain embodiments, the compositions described herein may also be used for improving bone, joint and muscle health.

In certain embodiments, the garlic extract components have a synergistic effect on improving (e.g. increasing) blood flow and/or inhibiting platelet aggregation.

In certain embodiments, the % inhibition of platelet aggregation by the composition or pharmaceutical composition is greater than the sum of the % inhibition of platelet aggregation by the individual garlic extract components present in the composition or pharmaceutical composition. For example, the % inhibition of platelet aggregation by the composition or pharmaceutical composition may be at least about 5% greater, for example at least about 10% greater, for example at least about 15% greater, for example at least about 20% greater, for example at least about 25% greater, for example at least about 30% greater (absolute value) than the sum of the % inhibition of platelet aggregation by the individual garlic extract components present in the composition or pharmaceutical composition. For example, the % inhibition of platelet aggregation by the composition or pharmaceutical composition may be up to about 100% greater (absolute value) than the sum of the % inhibition of platelet aggregation by the individual garlic extract components present in the composition or pharmaceutical composition.

In certain embodiments, the IC₅₀ of the composition or pharmaceutical composition is less than the expected IC₅₀ of the combination of individual garlic extracts in the composition. For example, the IC₅₀ of the composition or pharmaceutical composition is less than the expected IC₅₀ of the combination of the first and second garlic extracts. For example, the IC₅₀ of the composition may be at least about 5 μg/mL, for example at least about 10 μg/mL, for example at least about 15 μg/mL, for example at least about 20 μg/mL, for example at least about 25 μg/mL, for example at least about 30 μg/mL, for example at least about 25 μg/mL, for example at least about 35 μg/mL, for example at least about 40 μg/mL, for example at least about 45 μg/mL, for example at least about 50 μg/mL, less than the expected IC₅₀ of the combination of the individual garlic extract components. For example, the IC₅₀ of the composition may be up to about 300 μg/mL less than the expected IC₅₀ of the combination of the individual garlic extract components. In certain embodiments, the 10₅₀ of the composition or pharmaceutical composition may be less than the IC₅₀ of the garlic extract component (e.g. first or second garlic extract) with the lowest IC₅₀.

The expected IC₅₀ for the composition can be theoretically calculated using the following formula:

Expected IC₅₀=(A+B)/[(A/IC₅₀ of first garlic)+(B/IC₅₀ of second garlic)]

wherein A=absolute amount of first garlic in the mixture; and B=absolute amount of second garlic in the mixture.

In certain embodiments, the synergistic effect of the composition can be determined by Combination Index (CI), whereby CI<1 indicates synergy, CI=1 indicates additivity, CI>1 indicates antagonism. The formula for CI is:

CI=(X/IC₅₀ of first garlic)+(Y/IC₅₀ of second garlic)

wherein X=Concentration of first garlic in the mixture that produce 50% inhibition; and Y=Concentration of second garlic in the mixture that produce 50% inhibition.

The compositions and pharmaceutical compositions described herein may be used in various non-therapeutic applications.

For example, the compositions may be used in methods of maintaining blood flow in a subject. For example, where a subject is healthy or has a normal blood flow level, and may, for example, not be at any particular risk of developing any disease associated with blood flow and/or platelet aggregation, the subject may consume the compositions disclosed herein to maintain a normal blood flow level. For example, where a subject is healthy and may, for example, not be at any particular risk of developing any disease associated with blood flow and/or platelet aggregation, the subject may consume the compositions disclosed herein as part of a healthy lifestyle.

In some embodiments, the compositions described herein may be used to improve energy levels in a subject, e.g. by increasing or boosting energy levels. For example, the compositions described herein may be used to reduce or eliminate fatigue in a subject. For example, the compositions described herein may be used to improve endurance of a subject (e.g. during exercise (where a subject's heart rate increases to above their normal resting heart rate)). Endurance may be measured, for example, by measuring the total amount of exercise the subject can do in a fixed amount of time (e.g. total distance a subject can run) or by measuring the total time it takes a subject to do a fixed amount of exercise (e.g. time take to run a set distance). Any difference in endurance before and after the composition is consumed indicated whether there has been an improvement. This may be determined, for example 5 hours after, for example 12 hours after, for example 16 hours after, for example 24 hours after, for example 48 hours after, for example 3 days after, for example 4 days after, for example 7 days after, for example 14 days after, for example 21 days after, for example 28 days after, for example 35 days after, for example 42 days after, for example 48 days after, for example 56 days after the composition is consumed for the first time. The composition may be consumed by the subject at regular intervals between up until the difference in endurance is measured.

For example, the compositions described herein may be used to improve recovery time of a subject (e.g. after exercise). Recovery time may be considered to be the time it takes for the heart rate of an individual to return to their normal resting heart rate after exercise. Alternatively or additionally, recovery time may be considered to be the time it takes for lactic acid or lactate to be eliminated from the blood.

In another example, the compositions and pharmaceutical compositions described herein may be used to improve (e.g. increase) nutrient delivery to cells or tissues and/or to improve (e.g. increase) metabolic waste removal from cells or tissues (e.g. assists in the detoxification process).

In certain embodiments, the compositions described herein may also be used for cell protection, repair and regeneration. The compositions can also be used for anti-aging (e.g. the maintenance of good health during aging for example by providing healthy blood circulation to repair or regenerate aging cells).

The compositions and pharmaceutical compositions disclosed herein may be used in various therapeutic applications. For example, the compositions and pharmaceutical compositions disclosed herein may be used to improve (e.g. increase) blood flow and/or decrease platelet aggregation in a subject, for example in a subject that has a blood flow level that is outside what is considered to be a normal or healthy range. For example, the compositions and pharmaceutical compositions disclosed herein may be used to treat or prevent any disease or disorder associated with blood flow (e.g. circulation system) and/or platelet aggregation in a subject. For example, the subject may be susceptible to developing one or more diseases or disorders associated with blood flow and/or platelet aggregation.

The expression “treating or preventing” and analogous terms used herein refers to all forms of healthcare intended to remove or avoid the disorder or to relieve its symptoms, including preventive and curative care, as judged according to any of the tests available according to the prevailing medical practice. An intervention that aims with reasonable expectation to achieve a particular result but does not always do so is included within the expression “treating or preventing”. An intervention that succeeds in slowing or halting progression of a disorder is included within the expression “treating or preventing”.

The expression “susceptible to” and analogous terms used herein refers particularly to individuals at a higher than normal risk of developing a disease, for example, cardiovascular disease, cerebrovascular or brain disease, immune disease and/or bone, joint and/or muscle disease, as assessed using the known risk factors for the individual or disease, e.g., low blood flow, increased platelet aggregation, high cholesterol, high blood pressure, smoking, diabetes, sedentary lifestyle, obesity and genetic factors. Such individuals may, for example, be categorized as having a substantial risk of developing the disease to the extent that medication would be prescribed and/or special dietary, lifestyle or similar recommendations would be made to that individual.

Diseases and disorders associated with blood flow and/or platelet aggregation include, for example, cardiovascular diseases, cerebrovascular or brain disease, immune diseases, bone, joint and/or muscle diseases and fatigue diseases.

Cardiovascular diseases and disorders include, for example, coronary artery diseases, coronary heart disease, angina, myocardial infarction, hypertensive heart disease, heart failure, pulmonary heart disease, cardiac dysrhythmias, inflammatory heart disease, rheumatic heart disease, cardiomyopathy, atrial myopathy, congenital heart disease, endocarditis, inflammatory cardiomegaly, myocarditis, valvular heart disease, aortic aneurysms, venous thrombosis, rheumatoid vasculitis, atherosclerosis peripheral artery diseases and renal artery stenosis.

Cerebrovascular diseases and brain diseases include, for example, stroke (e.g. mini-stroke, hemorrhagic stroke, ischemic stroke), transient ischemic attack (TIA), subarachnoid haemorrhage and vascular dementia.

Immune diseases and disorders include, for example, any disease in which the subject's immune response is lower than a normal healthy individual, for example immunodeficiency diseases (e.g. primary, secondary, humoral, T cell, neutropenia, asplenia and complement deficiency diseases), low immune response in subjects whose immune system has been compromised (e.g. subjects undergoing chemotherapy and/or radiotherapy, subjects infected with HIV, subjects missing one or more organs associated with immune function such as the spleen, tonsils, lymph nodes). For example, immune diseases include ataxia-telangiectasia, Chediak-Higashi syndrome, combined immunodeficiency disease, complement deficiencies, DiGeorge syndrome, hypogammaglobulinemia, Job syndrome, leukocyte adhesion defects, panhypogammaglobulinemia, Bruton's disease, congenital agammaglobulinemia, selective deficiency of IgA, Wiskott-Aldrich syndrome and severe combined immunodeficiency disorder (SCID). For example, infection or symptoms related to immune health include upper and lower respiratory tract infection, hay fever, sinus and pharyngitis.

Joint diseases and disorders include, for example, any disease related to joints, mobility, muscle and bone health, for example arthritis (e.g. osteoarthritis, rheumatoid arthritis, psoriatic arthritis, septic arthritis), bursitis, osteonecrosis, dislocations, Perthes disease and Paget's disease of the bone.

Fatigue diseases and disorders include, for example, simple fatigue and/or any disease in which fatigue is a symptom. For example, fatigue diseases and disorders include chronic fatigue syndrome, anaemia, depression, iron deficiency (without anaemia), sleep disorders, underactive thyroid, overactive thyroid, Addison disease, anorexia nervosa or other eating disorders, autoimmune diseases such as lupus, diabetes, fibromyalgia, kidney disease, liver disease and malnutrition.

In certain embodiments, the subject is a human. In other embodiments, the subject is a mammal other than a human, such as non-human primates (e.g. apes, monkeys and lemurs), companion animals such as cats or dogs, working and sporting animals such as dogs, horses and ponies, farm animals such as pigs, sheep, goats, deer, oxen and cattle, and laboratory animals such as rodents (e.g. rabbits, rats, mice, hamsters, gerbils or guinea pigs).

The properties of the compositions and pharmaceutical compositions disclosed herein (e.g. the ability to improve (e.g. increase) blood flow and/or the ability to reduce platelet aggregation and/or the ability to improve (e.g. increase) nutrient delivery and/or the ability to remove metabolic waste) may be determined in vivo or in vitro. An in vitro method for determining the % inhibition of platelet aggregation of the compositions of the present invention is described in the Examples section below. Platelet aggregation may also be measured in vivo using, for example, radiolabelled platelets and external detection probes. Blood flow may, for example, be measured in vivo using ultrasonic or electromagnetic flow probes or sensors. Alternatively, blood flow may, for example, be measured using laser-doppler, infrared, light spectroscopy, ultrasound, flow-mediated vasodilation, peripheral arterial tone (PAT), pulse wave analysis, plethysmography, magnetic resonance imaging, positron emission tomography scanning or computer tomography (CT) scanning.

The IC₅₀ of the total garlic extracts in the composition (in terms of % inhibition of platelet aggregation) may, for example, be equal to or less than about 1000 μg/mL. For example, the IC₅₀ of the total garlic extracts in the composition may be equal to or less than about 900 μg/mL, for example equal to or less than about 800 μg/mL, for example equal to or less than about 700 μg/mL, for example equal to or less than about 600 μg/mL.

The platelet aggregation inhibitory effect of the composition and/or pharmaceutical composition can be measured using different types of test mimicking different mechanisms of the platelet aggregation, for example, by using different types of agonists as the indicators, such as arachidonic acid, thromboxane, thrombin, adenosine diphosphate, epinephrine and collagen. An example of the platelet aggregation test is detailed in Example 1, in which arachidonic acid is used as an agonist.

The compositions and pharmaceutical compositions described herein may be prepared by combining a first garlic extract and a second garlic extract and optionally any one or more of the other ingredients described herein, such as one or more further garlic extracts, dietary fibre, nutrients, biologically active agents and pharmaceutical excipients and/or carriers and/or diluents. The components are combined in suitable amounts to obtain a composition having the desired quantity of each component. Each component may be combined with one or more other components in any order and combination suitable to obtain the desired product. For example, each component may be combined by mixing (e.g. the first and second garlic extracts may be combined by mixing). Such methods are well known in the art, for example, methods known in the food industry (e.g. those used in the preparation of health food bars and the like) and methods known in the pharmaceutical industry. The composition may be prepared in the dry solid form, for example, powder form, and subject to further processing step depending on the types of the formulation for the intended finished products. The methods may further comprise a forming step, wherein the mixture is moulded, pressed, spray dried or otherwise formed into a shape (e.g. bar, ball, pellet, clusters, tablet), preferably with dimensions and/or textures suitable for consumption by a human or other mammalian animal of the types described herein.

The invention will now be described in detail by way of reference only to the following non-limiting examples.

### EXAMPLES

The inhibitory effect of various samples on platelet aggregation induced by arachidonic acid was determined in vitro. In this test human whole blood is centrifuged to obtain platelet rich plasma (PRP) (about 6×10⁸ platelets per mL). The supernatant platelet rich plasma is subjected to optimal aggregation by arachidonic acid at 37° C. as measured by an optical aggregometer.

The test was carried out in duplicate. Each cuvette contained PRP. The following test samples (water, indomethacin and test compounds) were added into the cuvettes respectively and incubated for 5 minutes. Subsequently, arachidonic acid was added and the test solution was further incubated for 5 minutes.

1. Water (solvent) was used as negative control to establish a maximal aggregation response.

2. Indomethacin was used as positive control to obtain an inhibitory response of arachidonic acid-induced platelet aggregation.

3. Test compounds were tested in a number of concentrations (for example 8 mg/mL, 4 mg/mL, 1 mg/mL, 500 μg/mL, 300 μg/mL) to obtain aggregation values for the determination of IC₅₀.

The samples that were tested are indicated in Table 1 below.

TABLE 1

| Sample | Concentration of Components |
|---|---|
| IQP-J | Deodourised garlic powder extract |
| | (DGPE) |
| IQP-K | Black garlic powder extract (BGPE) |
| IQP-A | IPQ-J + IPQ-K (2:1) |
| (Negative control) | Solvent (water) |
| (Positive control) | Indomethacin |

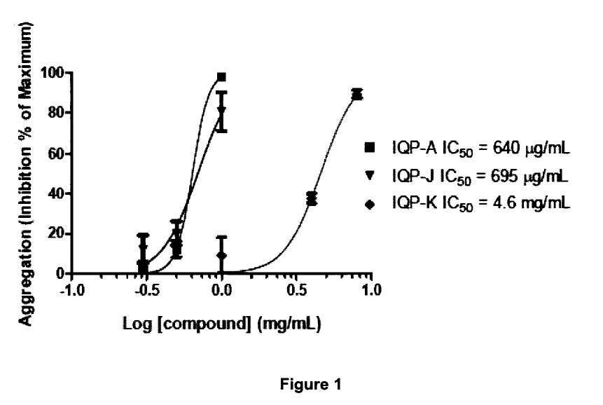

The results of the platelet aggregation test are shown in Table 2 below and FIG. 1 1.

TABLE 2

| Sample | IC₅₀ (μg/mL) |
|---|---|
| IQP-J | 695 |
| IQP-K | 4600 |
| IQP-A | 640 |

The IC₅₀ data of IQP-J, IQP-K and IQP-A was plotted and illustrated in FIG. 1. The expected inhibition of sample IQP-A (theoretically, a combination between samples of IQP-J and IQP-K in 2:1 ratio) can be derived from the inhibition graph of FIG. 1 based on the IC₅₀ values in Table 2.

As the IC₅₀ value of IQP-A is 640 μg/mL, it would be expected that the individual concentrations of the garlic components in sample IQP-A would be 427 μg/mL IQP-J and 213 μg/mL of IQP-K.

TABLE 3

| Sample | Concentration | Inhibition (%) |
|---|---|---|
| IQP-J | 427 μg/mL | 14 |
| IQP-K | 213 μg/mL | 0 |

Table 3 shows that the expected inhibition percentages of IQP-J and IQP-K, i.e. 14% and 0%, respectively. Accordingly, the sum of these values (14+0=14%) expected for IQP-A, is surprisingly lower than the actual platelet inhibition percentage that was obtained (50%) at this concentration of sample IQP-A. Therefore, the combination of sample IQP-J and sample IQP-K surprisingly has a synergistic effect on inhibition of platelet aggregation.

Additionally, the synergistic effect can be determined by the CI formula as described in one of the embodiments of the present invention:

$$\begin{matrix}C I of the composition ( IQP \text{-} A ) = ( 427 / 695 ) + ( 213 / 4600 ) \\ = 0.66\end{matrix}$$

As CI is <1, the composition is shown to have synergistic effect on inhibition of platelet aggregation.

1. A composition comprising:
a first garlic extract; and
a second garlic extract different to the first garlic extract.

2. A composition according to claim 1, wherein the first and/or second garlic extract, is derived from raw garlic, aqueous garlic extract, non-aqueous garlic extract, alcoholic garlic extract, garlic concentrate, garlic oil, garlic maceration, garlic powder, garlic granules or any combination of two or more thereof.

3. A composition according to claim 1, wherein the first garlic extract and the second garlic extract have a synergistic effect on inhibition of platelet aggregation.

4. A composition according to claim 3, wherein a % inhibition of platelet aggregation by the composition is greater than a sum of individual % inhibition of platelet aggregation by the first and second garlic extracts.

5. A composition according to claim 4, wherein the % inhibition of platelet aggregation by the composition is at least about 10% greater than the sum of the individual % inhibition of platelet aggregation by the first and second garlic extracts.

6. A composition according to claim 1, wherein a half maximal inhibitory concentration (IC₅₀) of the composition is less than an expected IC₅₀ of the combination of the first and the second garlic extract.

7. A composition according to claim 6, wherein the IC₅₀ of the composition is less than the IC₅₀ of the first or second garlic extract with the lowest IC₅₀.

8. A composition according to claim 1, wherein one of the first and the second garlic extracts is an aged garlic extract.

9. A composition according to claim 8, wherein the aged garlic extract is a black garlic extract, caramelised garlic extract, fermented garlic extract, and/or black garlic powder extract (BGPE).

10. A composition according to claim 1, wherein one of the first and the second garlic extracts is a non-aged garlic extract.

11. A composition according to claim 10, wherein the non-aged garlic extract is deodourized garlic extract or deodourized garlic powder extract (DGPE).

12. A composition according to claim 1, wherein the first garlic extract is a black garlic extract and the second garlic extract is a deodourized garlic extract.

13. A composition according to claim 1, wherein the weight ratio of the first garlic extract to the second garlic extract ranges from 1:10 to 10:1.

14. A composition according to claim 1, wherein the first garlic extract is a black garlic extract and the second garlic extract is a deodourized garlic extract, and the weight ratio of the first to the second garlic extract is about 1:2 or about 3:7.

15. A composition according to claim 1, wherein the composition comprises allicin, polyphenol, alliin, γ-glutamylcysteine, S-allyl-L-cysteine, thiosulfinates, sulfur compounds or any combination thereof.

16. A composition according to claim 1, wherein the composition comprises:
a) equal to or greater than about 0.5% (w/w) allicin; and/or
b) equal to or greater than about 0.5% (w/w) polyphenol; and/or
c) equal to or greater than about 0.5% (w/w) total thiosulfinates; and/or
d) equal to or greater than about 1.5% (w/w) alliin; and/or
e) equal to or greater than about 1.5% (w/w) γ-glutamylcysteine; and/or
f) equal to or greater than about 0.3% (w/w) total sulfur; and/or
g) equal to or greater than about 0.05% (w/w) S allyl-L-cysteine.

17. A composition according to claim 1, further comprising a third garlic extract different to each of the first and second garlic extracts.

18. A composition according to claim 1, wherein the composition is a foodstuff, a food supplement, a beverage, a beverage supplement, a dietary supplement, a health supplement, a meal replacement product, a food additive, an animal feed or a feed additive.

19. A pharmaceutical composition comprising a composition according to claim 1 and a pharmaceutically acceptable excipient and/or carrier and/or diluent.

20. A composition according to claim 1, for use in improving or increasing blood flow in a subject and/or for use in reducing platelet aggregation in the subject and/or for use in maintaining and/or improving the overall health of the circulatory system in the subject.

21. Use of a composition according to claim 1 in the manufacture of a medicament for improving or increasing blood flow in a subject and/or for reducing platelet aggregation in the subject and/or for maintaining and/or improving the overall health of the circulatory system in the subject.

22. A therapeutic method for improving or increasing blood flow in a subject and/or for reducing platelet aggregation in the subject and/or for maintaining and/or improving the overall health of the circulatory system in the subject, the method comprising administering a composition according to claim 1 to the subject.

23. A non-therapeutic method for maintaining or improving or increasing blood flow in a subject and/or for reducing platelet aggregation in the subject and/or for maintaining and/or improving the overall health of the circulatory system in the subject, the method comprising administering a composition according to claim 1 to the subject.

24. A composition according to claim 1, wherein the composition is for treating or reducing or preventing the onset of a cardiovascular disease, for treating or reducing or preventing onset of a cerebrovascular or brain disease, for treating or reducing or preventing onset of an immune disease, for treating or reducing or preventing onset of a bone, joint and/or muscle disease, for increasing energy and/or reducing or preventing onset of fatigue in a subject, for improving nutrient delivery and/or metabolic waste removal, or for reducing or preventing cell oxidation and/or improving cell protection and repairing activities.

25-31. (canceled)
